(19) United States
(12) Patent Application Publication
(10) Pub. No.: US 2011/0014132 A1
(43) Pub. Date: Jan. 20, 2011

(54) SOLID EFFERVESCENT MIXTURE FOR THE ORAL ABSORPTION

(76) Inventor: Shuang Peter Liu, Bridgewater, NJ (US)

Correspondence Address: Progressive IP Solutions, 32 Pershing Street, Staten Island, NY, 10305, US

(21) Appl. No.: 12/833,952

(22) Filed: Jul. 10, 2010

Related U.S. Application Data
(60) Provisional application No. 61/270,842, filed on Jul. 14, 2009.

(51) Int. Cl. A61K 9/12; A61P 25/34; B05D 3/00

(52) U.S. Cl. 424/43; 427/2.14

(57) ABSTRACT

The present invention is a solid effervescent mixture for oral absorption which comes in the following forms: tablets, pills, granules, chewing gum, and candy. The formulation and preparation methods of the solid effervescent mixture are also disclosed. The solid effervescent formulation is administrated orally without water for dissolving and swallowing. In general, the present invention is directed to a solid or other mixture that comprises an orally administrable therapeutic agent or agents, in combination with an effervescent formulation that may be used as an absorption enhancer for the therapeutic agents across the buccal, sublingual, and/or gingival mucosa in order to relieve local and systemic symptoms rapidly.

### RELATED APPLICATION

This application claims the benefit of U.S. Provisional Application No. 61/270,842 filed Jul. 14, 2010, which is incorporated herein by reference.

### BACKGROUND OF THE INVENTION

The present application is directed to a solid or other mixture that includes an orally administrable therapeutic agent or agents, in combination with an effervescent formulation that may be used as an absorption enhancer for the therapeutic agents across the buccal, sublingual, and/or gingival mucosa.

U.S. Pat. No. 5,962,022 assigned to SmithKline Beecham (GB) describes an invention that relates to pharmaceutical compositions for oral administration of antibiotics and other medicament with unpleasant taste characteristics and particularly to compositions formulated as tablet. This chewable composition was especially suitable for improving the taste characteristics of a range of medicaments, particularly for improving the taste of bitter tasting medicaments and also provided a pleasant mode of administrating medicaments, particularly those that result in an unpleasant mouth feel even in the absence of a bitter taste. e.g., antacids. The effervescent couple comprises a basic ingredient and an acidic ingredient. The basic ingredient liberates carbon dioxide when it comes in contact with acidic ingredient and saliva or added to water. The effervescent couple typically includes citric acid or sodium hydrogen citrate and sodium bicarbonate. Another aspect of this invention is incorporation of disintegrants that gives the patient an option of dispersing the tablet in small amount of water prior to administration.

U.S. Pat. No. 4,639,368 assigned to Farmacon Research Corporation (MN) describes a chewing gum containing a medicament capable of absorption through the buccal cavity and containing a taste masking amount of an effervescent. Effervescent compositions have also been employed for use as taste masking agents in dosage forms which are not dissolved in water prior to administration. Gums also leave residues which must be disposed of.

U.S. Pat. No. 6,245,353 assigned to Asta Medica AG (DE) describes an invention that provides a novel and therapeutically advantageous solid, rapidly disintegrating, effervescent, rapidly dissolving dosage form for oral administration of cetrizine. The dosage form contains organic edible acid, alkali metal and an alkaline earth metal carbonate and bicarbonate and optionally a pharmaceutically acceptable auxiliary ingredient along with the drug. The formulation of this invention when dissolved in water yields a solution having a pleasant taste with improvement in patient compliance. This obviates the tedious need of coating the individual crystal of cetrizine for masking the bitter taste. The patent describes the first ever effervescent preparation of cetrizine, which is very effective against allergic disorders.

U.S. Pat. No. 6,242,002 assigned to Arzneimittelwerk Dresden GmbH (DE) describes a rapidly disintegrating form of oral dosage. Patients suffering from Parkinsons disease usually have problem when swallowing a tablet with a liquid due to strong tremors. Also, administration of tablet for a patient having swallowing difficulty is not possible. The object of rapid disintegration of Selegiline (an antipakinsonian drug) was achieved by rapidly disintegrating oral dosage form (with or without water) as an effervescent formulation comprising an alkali sensitive drug and an effervescent base of an alkaline earth metal carbonate, an organic edible acid and an alkali metal salt of citric acid and optionally, a pharmaceutically acceptable auxiliary ingredient. This invention discloses an effervescent formulation that can be in the form of granules or tablets. The tablet can also be buccal tablet. By adding water or bringing in contact such effervescent formulation with saliva, a suspension or solution with carbon dioxide evolution results and such suspension or solution has a pleasant taste. It also aids in rapid release of ingredients.

More recently effervescents have been employed to obtain rapid dissolution and/or dispersion of the medicament in the oral cavity. See U.S. Pat. Nos. 5,178,878 and 5,223,264. The effervescent tends to stimulate saliva production thereby providing additional water to aid in further effervescent action. These dosage forms give an agreeable presentation of the drug, particularly for patients who have difficulty in swallowing tablets or capsules. PCT application WO 97/06786 describes pre-gastric absorption of certain drugs using rapidly-disbursing dosage forms.

Various proposals have been advanced for oral mucosal administration of various drugs. When drugs are absorbed from the oral mucosa, they bypass the gastrointestinal and hepatic metabolism process. This can lead to a faster onset of action and/or improved bioavailability of a drug. However, many compounds do not rapidly penetrate the oral mucosa. See, e.g., Christina Graffner, Clinical Experience with Novel Buccal and Sublingual Administration; NOVEL DRUG DELIVERY AND ITS THERAPEUTIC APPLICATION, edited by L. F. Prescott and W. S. Nimmo (1989); David Harris & Joseph R. Robinson, Drug Delivery via the Mucous Membranes of the Oral Cavity; JOURNAL OF PHARMACEUTICAL SCIENCES, Vol. 81 (January 1992); Oral Mucosal Delivery, edited by M. J. Rathbone, which are herein incorporated by reference. The compounds which may be well absorbed per-orally (through the gastrointestinal tract) may not be well absorbed through the mucosa of the mouth because the oral mucosa is less permeable than the intestinal mucosa and it does not offer as big a surface area as the small intestine.

Despite these and other efforts toward increasing the permeation of medicaments across the oral mucosa, there have been unmet needs for improved methods of administrating medicaments across the oral mucosa.

### SUMMARY OF THE INVENTION

In general, the present invention is directed to a solid or other mixture that comprises an orally administrable therapeutic agent or agents, in combination with an effervescent formulation that may be used as an absorption enhancer for the therapeutic agents across the buccal, sublingual, and/or gingival mucosa in order to relieve local and systemic symptoms rapidly.

In a more preferred embodiment, the solid or other mixture comprises additionally at least one component selected from a group consisting of one or more therapeutic agents, processing materials, excipient including sweeteners, edible oils, flavoring and coloring agents, processing aids, and/or breath fresheners. The formulation and preparation methods of the solid effervescent mixture are also disclosed.

In a more specific embodiment, this invention relates to a solid mixture effervescent tablet, pill, granule, candy and/or chewing gum, wherein the solid mixture is an energy enhancing effervescent tablet or other form, and/or an oral health effervescent mint, a nicotine replacement effervescent tablet and/or a sober up effervescent tablet.

Additional aspects of the present invention will be apparent in view of the description which follows.

### BRIEF DESCRIPTION OF THE FIGURES

FIG. 1 is a flow diagram for processing effervescent tablets, pills, and granules according to at least one embodiment of the methods disclosed herein;

FIG. 2 is a flow diagram for processing effervescent chewing gums, and candy according to at least one embodiment of the methods disclosed herein.

FIG. 3 is a chart showing the comparison of alcohol concentration in blood with and without sober up effervescent tablet according to at least one embodiment of the tablets disclosed herein.

FIG. 4 is a chart showing the comparison of caffeine concentration in blood taken according to at least one of the methods disclosed herein and orally.

FIGS. 5-6 are a chart showing the effect of oral bacteriostasis by comparing different dosage intake of the tablets according to one embodiment of the tablets disclosed herein.

FIG. 7 is a chart showing the effect on dispel halitosis by taking one oral effervescent tablet according to at least one embodiment of the tablets disclosed herein.

### DETAILED DESCRIPTION OF THE INVENTION

In at least one embodiment, a solid effervescent mixture for oral absorption is provided, such as tablets, pills, granules, candy and edible chewing gums, that includes therapeutic agents, basic components, acidic components, processing materials and an excipient. The formula for the solid mixture includes: Basic components in an amount of about 5% to about 50%; Acidic components in an amount of about 5% to about 50%; Therapeutic agents in an amount of about 1% to about 40%; Processing materials in an amount of about 0.5% to about 35%, and Excipient in an amount of about 1% to about 10%, or preferably includes: Basic components in an amount of about 10% to about 30%; Acidic components in an amount of about 10% to about 30%; Therapeutic agents in an amount of about 5% to about 30%; Processing materials in an amount of about 0.5% to about 35%; and Excipient in an amount of about 1% to about 10%.

The solid effervescent mixture for the oral absorption according to at least one embodiment provides a convenient, fast and safe administration method, and there is no need for water to help dissolving and swallowing. After being orally administered, e.g., by placing the solid mixture under the tongue, the solid effervescent mixture will rapidly dissolve with saliva and will produce a large amount of bubbles and water.

Traditional solid effervescent preparations are usually produced in a tablet form with an appropriate acid and a base as disintegrate agent. Before administration, the base is usually dissolved in water, which produces carbon dioxide. Once dissolved, the solid effervescent is then orally administrated and absorbed in gastrointestinal tract. Traditional effervescent preparations therefore are absorbed to blood circulation system via gastrointestinal tract in order to take effect. The onset time is normally slow, requiring more than 30 minutes to take effect. Moreover, before the drug enters the circulation system, the first-pass elimination of the liver and the stimulus to gastrointestinal mucosa by the drug, various enzymes in bile and gastrointestinal tract will affect the absorption and bioavailability of the drug, decrease the dosage of drug entering blood circulation system, and then result in poor drug efficiency and bioavailability.

To prevent the disadvantages of traditional orally administrated medicaments, the solid effervescent mixture for the oral absorption according to at least one embodiment of the invention provides a convenient, fast and safe administration method. There is no need for water to help in dissolving and swallowing. Because sublingual mucosa has high diffusion ability the therapeutic agent in the solid effervescent mixture will rapidly absorbed into blood circulation system, it greatly improves absorption rate and maximizes the bioavailability of therapeutic agent in the solid effervescent mixture, and improves the absorption and curative effect of therapeutic agent to relieve local and systemic symptoms. Moreover, the present invention avoids the liver's first pass elimination and stimulus to gastrointestinal tract of traditional effervescent tablets.

The solid effervescent mixture for oral absorption according at least one embodiment contains the formulation at least one processing material such as PEG (polyethylene glycol), PVP (polyvinylpyrrolidone), panthenol, vitamin E acetate, glycerin monostearate and their mixture. The processing material can be used in wet granulation or melt agglomeration, and acts as binder and/or enclosed materials, and can encapsulate the basic component, acidic component, therapeutic agents, to improve stability and the dissolution rate of the solid effervescent mixture. Moreover, by coupling different molecular weight of PEG with water-insoluble therapeutic agents, such as Chinese herb extract, the processing material can increase solubility of the therapeutic agents. PEG as disintegrants blend with different ratios of basic component, acidic component, make the mixture have better non-hygroscopic, lubricating performance, binding effect, and disintegration. Moreover, it enhances the speed and degree of effervescent effect of the present invention the solid effervescent mixture

The following preparation methods further illustrate the invention.

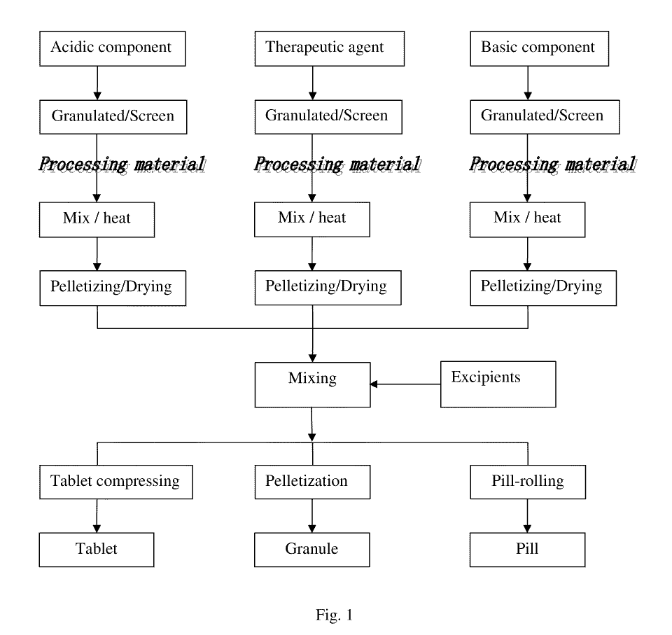

The preparation process of solid effervescent mixture the tablets, pills and granule, the preparation process of solid effervescent tablets, pills and granule dosage forms, are made from therapeutic agents, acidic components and basic components. The components are granulated and passed by 80 mesh screen separately. According to the solid formulation, processing materials is added into proportional amount of therapeutic agents, acid components and basic components also separately. Processing material such as PEG is added by melt agglomeration or PVP-ethanol by wet granulation to each of the therapeutic agents, acid components, and basic components separately, melted and dried in an 85° C. oven, and then cooled to room temperature. Each is then processed via pelletization and sorted through a 20 mesh screen, as shown in FIG. 1. According to solid formulation, appropriate amounts of therapeutic agents, acidic components and basic components are mixed together, and according to the different product requirement, appropriate amount of excipient such as sweeteners, flavoring and coloring agents, processing aids are added to make the mixture into tablets, pills and granule.

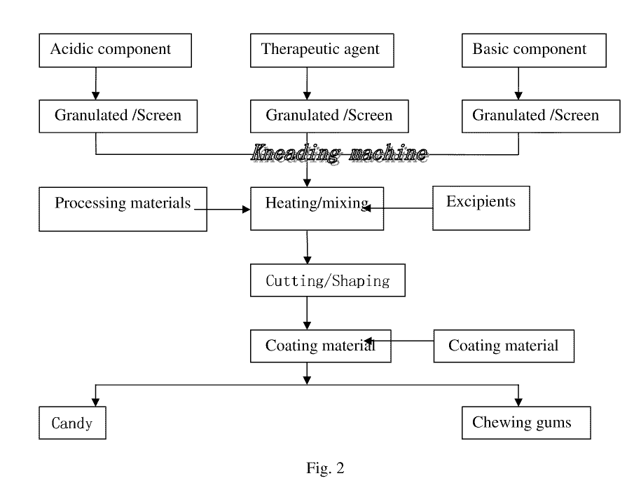

The solid effervescent mixture in the form of a chewing gum and candy are made from therapeutic agents, acidic components and basic components. The components are granulated and passed by 80 mesh screen separately. According to solid formulation, in proportional amount of therapeutic agents, acid components and basic components are taken and mixed together with processing material, such as panthenol and glycerin monostearate in a kneading machine. The mixture is then heated to 90° C. and the melted mixture is mixed while adding appropriate amounts of an excipient(s), such as sweeteners, flavoring and coloring agents, processing aids. The mixture is then turned into a softwood sheet, according to the different product requirement, and cut and shaped, and placed into a coating machine that add coating materials to make chewing gum and candy, as shown in FIG. 2.

1. Description of Sober Up (Alcoholism) Effervescent Products

There are many sober up products on the market, like tablet, capsule, tea and liquid. They are not easy to carry and need water for administration, the absorption of the active component in gastrointestinal tract will be reduced, and the concentration by first-pass elimination of liver decreased. Moreover, these products take a longer time to sober up and have a lesser effect—especially if the patient is unable to swallow when drunk or passed out.

The present sober up effervescent products according to one embodiment contains therapeutic agents that are sober up (alcoholism) and detoxifying components, such as Chinese herb extract from milk thistle seed, hawthorn fruit, green tea, hovenia dulcis, nutmeg, poria cocos, fructus tsaoko, radix puerariae, semen ziziphi spinosae, ramulus cinnamoni, alpinia officinarum hance, and chrysanthemum. When one takes sober up effervescent tablet before and after drinking, the therapeutic compositions are absorbed by buccal mucosa to sober up effectively, and to detoxify alcohol's damages to liver. By effervescent effect the composition stimulates the metabolism of alcohol and lowers the alcohol concentration in blood circulation quickly. By sublingual administration the absorption rate and bioavailability of therapeutic component is second only to injection and inhalation. Sober up effervescent tablet requires no water to be in effect and therefore is especially fit for patients who have difficulty swallowing. The table allows users to sober up rapidly and also refresh breath at the same time, while being easy to carry and to use any time in any event.

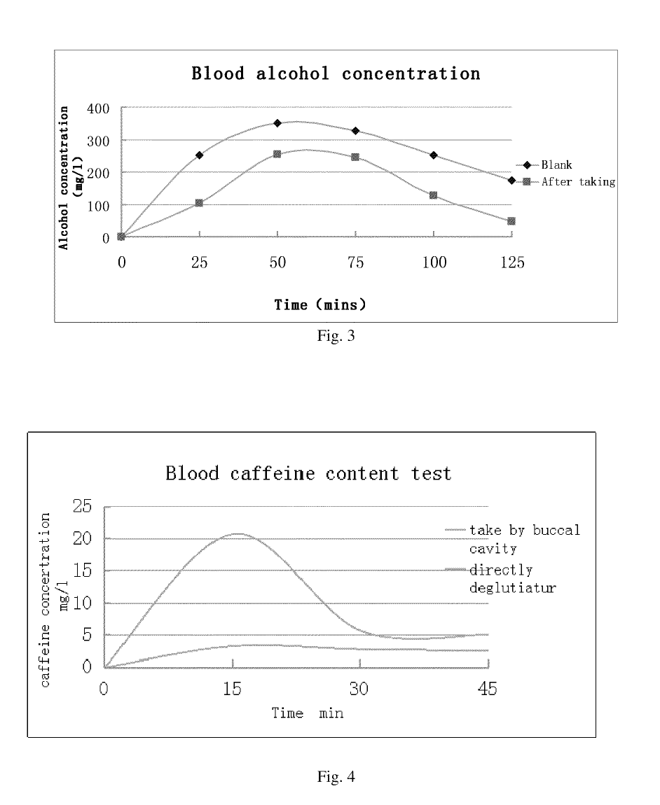

After human subjects drank 100 ml of white wine, their blood was tested every 25 minutes for 1.5 hours. Their resulting blood alcohol concentration with and without sober up effervescent tablets intake is shown in FIG. 3. The difference in the lines is shown that the alcohol concentration in the blood decreased rapidly by taking 2 (1.2 g dosage) sober up effervescent tablets before and after drinking. Another test was also performed where two cups filled with 50 ml white wine (53% alcohol concentration). The one with sober up effervescent tablets dissolved therein was unable to light on fire whereas the one without sober up tablet could be lit on fire.

The following examples are to illustrate the preparation method of sober up effervescent products including tablets, pills, granules, chewing gums, candy, but preparation methods are not limited to these.

### EXAMPLE

Example 1, citric acid 5%, sodium bicarbonate 35%, calcium carbonate 15%, vitamin C 0.2%, glucurolactone 0.3%, green tea extract 0.5%, PEG 35%, aspartame 0.3%, mint flavor 0.6%, sorbitol 4%, micro silicon 1%, and colorant.

Example 2, malic acid 50%, sodium carbonate 5%, glucurolactone 5%, hawthorn fruit extract 35%, PEG 0.5%, stevioside 0.3%, hawthorn powder 0.9%, PVP 3%, dolomol 0.15%, and colorant.

Example 3, tartaric acid 10%, calcium carbonate 30%, vitamin C 10%, hovenia dulcis extract 10%, panthenol 16%, PEG 15%, spicy powder 0.8%, mannitol 7%, stevioside 0.2%, talc powder 0.95%, and colorant.

Example 4, fumaric acid 10%, citric acid 20%, baking soda 5%, pearl powder 5%, vitamin C 5%, nutmeg extract 20%, PEG 5%, glycerin monostearate 20%, sucralose 0.1%, mint flavor 0.35%, dextrin 9.5%, and colorant.

Example 5, fumaric acid 7%, citric acid 10%, baking soda 10%, calcium carbonate 13%, pearl powder 5%, glucurolactone 10%, vitamin C 5%, radix puerariae extract 15%, PEG 12%, panthenol 7%, sucralose 0.2%, flavoring strawberry essence 0.25%, dolomol 0.5%, and colorant.

Example 6, citric acid 15%, folic acid 10%, pearl powder 35%, vitamin C 10%, fructus tsaoko extract 5%, glycerin monostearate 15%, orange powder 2%, saccharose 2%, lactose 5%, and colorant.

Example 7, citric acid 35%, calcium carbonate 40%, glucurolactone 2%, radix puerariae extract 3%, PEG 13%, hawthorn powder 4.5%, neotame 0.2%, avicel 2%, and colorant.

Example 8, maleic acid 15%, tartaric acid 25%, baking soda 45%, glucurolactone 8%, poria cocos extract 2%, panthenol 4%, aspartame 0.2%, almond powder 0.2%, talc powder 0.55%, and colorant.

Example 9, Tartaric acid 5%, adipic acid 10%, calcium carbonate 12%, baking soda 8%, vitamin C 2%, glucurolactone 13%, spine date seed extract powder 20%, PEG22%, stevioside 0.2%, mint flavor 0.1%, sorbitol 7%, micro silicon 0.65%, and colorant.

Example 10, calcium hydrogen phosphate 45%, baking soda 5%, pearl powder 10%, semen ziziphi spinosae 20%, PEG 10%, sucralose 0.3%, flavoring strawberry essence 0.5%, glycerin 6%, gelatin 3%, and colorant.

Example 11, malic acid 50%, sodium carbonate 5%, glucurolactone 40%, PEG 0.5%, stevioside 0.3%, hawthorn powder 1%, Vitamin E acetate3%, and colorant.

Example 12, maleic acid 5%, baking soda 30%, calcium carbonate 20%, vitamin C 1%, PEG 35%, aspartame 0.3%, mint flavor 0.6%, sorbitol 7%, edible oil 1%, and colorant.

Example 13, tartaric acid 15%, calcium carbonate 25%, vitamin C 4%, glucurolactone 4%, alpinia officinarum hance extract 12%, panthenol 15.5%, PEG 15%, glycerin 8%, sucralose 0.4%, flavoring lemon essence 0.6%, and colorant.

Example 14, fumaric acid 10%, malic acid 20%, pearl powder 5%, baking soda 25%, glucurolactone 20%, hawthorn fruit extract 5%, glycerin monostearate 5%, edible oil 5%, vitamin E acetate 1.5%, gelatin 3%, sucralose 0.1%, mint flavor 0.35%, and colorant.

Example 15, Tartaric acid 6%, citric acid 11%, baking soda 10%, calcium carbonate 13%, pearl powder 10%, vitamin C 5%, glucurolactone 10%, green tea extracts 15%, panthenol 1%, PEG 17.5%, vitamin E acetate 1%, neotame 0.2%, flavoring strawberry essence 0.25%, and colorant.

Example 16, citric acid 16%, tartaric acid8%, calcium carbonate 17%, pearl powder 8%, vitamin C 3%, glucurolactone 15%, hovenia dulcis extracts 5%, glycerin monostearate 18%, orange flavor 0.4%, saccharose 1%, glycerol 3.5%, gelatin 5%, and colorant.

Example 17, citric acid 35%, calcium carbonate 40%, radix puerariae extract 5%, PEG 10%, gelatin 8%, edible oil 1.5%, aspartame 0.3%, coffee powder 0.1%, and colorant.

Example 18, adipic acid 17%, tartaric acid 25%, baking soda 20%, glucurolactone 1%, fructus tsaoko extracts 12%, panthenol 15%, mannitol 9.5%, aspartame 0.2%, almond powder0.2%, and colorant.

Example 19, tartaric acid 10%, citric acid 5%, calcium carbonate 45%, vitamin C 8%, glucurolactone 7%, nutmeg extract 20%, PEG 4%, vitamin E acetate0.5%, stevioside 0.2%, mint flavor 0.2%, and colorant.

2. Description of Energy Enhancing Effervescent Products

Energy drinks are very popular around the world. Beverages such as Coca-Cola and Red Bull are energy drinks in cans, bottles and powders to be reconstituted with water, mostly based on caffeine with a little flavor, color, and a few vitamins and minerals. Coca-Cola and Red Bull contain a lot of sugar; moreover, there is 34 mg caffeine in every 12 oz Coca-Cola, 45 mg caffeine in Diet Coca-Cola. There is even more caffeine in Red Bull: 80 mg in every 8.2 oz of Red Bull. Hundreds of variations of Coca-Cola and Red Bull exist; most contain caffeine that is absorbed in the gastrointestinal tract by drinking. They are slow to take effect and have low bioavailability; and they are not convenient to carry and their packaging needs to be discarded.

To avoid the disadvantages of these energy drinks, the present invention according one embodiment, contains therapeutic agents such as natural caffeine, herbal extracts, vitamins and minerals. It comes in the solid form of energy enhancing effervescent mixture which is convenient to take without water and fast boost energy by oral absorption. Therapeutic agents are rapidly and directly absorbed into blood circulation system by sublingual and gingiva mucosa during effervescent reaction. It could avoid first-pass elimination and doesn't stimulate gastrointestinal tract; it takes effect much faster to recover and boost energy. The comparison of caffeine concentration in blood with energy enhancing effervescent tablets intake by oral absorption and gastrointestinal absorption is showed in FIG. 4.

In FIG. 4, one line represents the use of 3 tablets (90 mg caffeine) sublingually. The other line represents the use of 3 tablets when swallowed. For sublingual absorption, the concentration of caffeine in blood peaks at 15 minutes to more than four times the concentration of caffeine compared to gastrointestinal absorption.

The following examples are to illustrate the preparation method of energy enhancing effervescent products, but preparation methods are not limited to these.

### EXAMPLE

Example 1 provides a representative solid mixture formulation which is representative of compositions of the present invention; a sugar free, energy enhancing effervescent tablet that includes: a edible basic formulation that comprises at least one component selected from the group consisting of alkali metal carbonates, alkali metal bicarbonates, alkaline earth metal carbonates, alkaline earth metal bicarbonates, pearl powder and mixtures thereof; and an edible acid formulation that comprises at least one component selected from the group consisting of tartaric acid, citric acid, fumaric acid, maleic acid, malic acid, ascorbic acid, folic acid, acid phosphate salts, and mixtures thereof; wherein the edible basic formulation and the edible acid formulation are in the range of from about 5 to about 50 percent by weight of the oral health effervescent mint, and octacosanol in an amount of from about 0.1% to about 35%; glucuronolactone in an amount of from about 0.1% to about 35%; taurine in an amount of from about 0.1% to about 30%; natural caffeine in an amount of from about 0.1% to about 25%; rhodiola in an amount of from about 0.1% to about 25%; ginseng extract in an amount of from about 0.1% to about 25%; d-ribose in an amount of from about 0.1% to about 25%; polyethylene glycol in an amount of from about 1% to about 35%; at least one artificial sweetener; and at least one flavoring and coloring agents.

Method of Preparation:

Ingredients are mixed and are compressed into a tablet using a suitable effervescent system that includes a basic component (sodium bicarbonate, sodium carbonate, calcium carbonate, but not limited to these) with a suitable acidic component (malic, citric, tartaric, but not limited to these). Typically one uses a 1:1 ratio of basic components to acidic components and total amount of about 10% to about 30%. In addition, an amount of about 5% to about 30% of therapeutic agents are mixed in the formulation; to assist in tablet disintegration about 0.5% to about 35% of processing material must be used such as PEG, PVP. Ingredients are mixed then compressed using a suitable tablet press under conditions of low humidity—less than 35% relative humidity. Additional excipient such as sweeteners, flavoring and coloring agents should be used to help compression

The following examples are to illustrate the preparation method of energy enhancing effervescent including tablets, pills, granules, chewing gums, candy, but preparation methods are not limited to these.

Example 1: citric acid 5%, baking soda 35%, calcium carbonate 15%, d-ribose 0.2%, glucurolactone 0.3%, ginseng extract 0.5%, PEG 35%, aspartame 0.3%, mint flavor 0.6%, sorbitol 4%, micro silicon 1%, and colorant.

Example 2:malic acid 50%, sodium carbonate 5%, natural caffeine 5%, ginseng extract 35%, PEG 0.5%, stevioside 0.3%, hawthorn powder 0.9%, PVP 3%, dolomol 0.15%, and colorant.

Example 3: tartaric acid 10%, calcium carbonate 30%, octacosanol 10%, d-ribose 10%, panthenol 16%, PEG15%, spicy powder 0.8%, mannitol 7%, stevioside 0.2%, talc powder 0.95%, and colorant.

Example 4: fumaric acid 10%, citric acid 20%, baking soda 5%, pearl powder 5%, taurine 5%, d-ribose 15%, ginseng extract 5%, PEG 5%, glycerin monostearate 20%, sucralose 0.1%, mint flavor 0.35%, dextrin 9.5%, and colorant.

Example 5: tartaric acid 7%, citric acid 15%, baking soda 10.5%, calcium carbonate 17.5%, natural caffeine 2%, octacosanol 5%, ginseng extract 10%, d-ribose 13%, PEG 12%, panthenol 7%, sucralose 0.2%, flavoring strawberry essence 0.25%, dolomol 0.5%, and colorant.

Example 6: citric acid 10%, folic acid 15%, pearl powder 15%, taurine 10%, glucurolactone 5%, glycerin monostearate 15%, orange 2.9%, saccharose 2%, lactose 5%, and colorant.

Example 7: citric acid35%, calcium carbonate 40%, d-ribose 3%, ginseng extract 2%, PEG 13%, hawthorn powder 4.5%, neotame 0.2%, avicel 2%, and colorant.

Example 8: maleic acid 15%, tartaric acid 25%, baking soda 45%, natural caffeine 3%, taurine 7%, panthenol 4%, aspartame 0.2%, almond powder 0.2%, talc powder 0.55%, and colorant.

Example 9: tartaric acid 5%, adipic acid 10%, calcium carbonate 12%, baking soda 8%, octacosanol 2%, taurine 3%, d-ribose 5%, ginseng extract 25%, PEG 22%, stevioside 0.2%, flavoring lemon essence 0.1%, sorbitol 7micro silicon 0.65%, and colorant.

Example 10: calcium hydrogen phosphate 45%, baking soda 5%, pearl powder 10%, ginseng extract 15%, PEG 15%, sucralose 0.3%, flavoring strawberry essence 0.5%, glycerol 6%, gelatin 3%, and colorant.

Example 11: malic acid 50%, sodium carbonate 5%, d-ribose 40%, PEG 0.5%, stevioside 0.3%, hawthorn powder 1%, Vitamin E acetate 3%, and colorant.

Example 12: maleic acid5%, baking soda 30%, calcium carbonate 20%, natural caffeine 1%, PEG 35%, aspartame e0.3%, mint flavor 0.6%, sorbitol 7%, edible oil 1%, and colorant.

Example 13: tartaric acid 15%, calcium carbonate 25%, d-ribose 5%, glucurolactone 10%, ginseng extract 5%, panthenol 15.5%, PEG 15%, glycerol 8%, sucralose 0.4%, flavoring lemon essence 0.6%, and colorant.

Example 14: fumaric acid 10%, malic acid 20%, pearl powder 5%, baking soda 25%, d-ribose 20%, natural caffeine 5%, glycerin monostearate 5%, edible oil 5%, vitamin E acetate 1.5%, gelatin 3%, sucralose 0.1%, mint flavor 0.36%, and colorant.

Example 15: tartaric acid 7%, citric acid 15%, baking soda 10%, calcium carbonate 13%, pearl powder 5%, natural caffeine 2%, taurine 2%, d-ribose 6%, ginseng extract 20%, panthenol 1%, PEG 17.5%, vitamin E acetate 1%, neotame 0.2%, flavoring strawberry essence 0.25%, and colorant.

Example 16: citric acid 10%, tartaric acid 15%, calcium carbonate 17%, pearl powder 18%, octacosanol 5%, glucurolactone 2%, d-ribose 3%, glycerin monostearate 20%, orange powder 0.4%, saccharose 1%, glycerol 3.5%, gelatin 5%, and colorant.

Example 17: citric acid 35%, calcium carbonate 40%, taurine 5%, PEG 10%, gelatin 8%, edible oil 1.5%, aspartame 0.3%, coffee powder 0.1%, and colorant.

Example 18: adipic acid 17%, tartaric acid 23%, baking soda 20%, octacosanol 3%, taurine 4%, d-ribose8%, panthenol 15%, mannitol 9.5%, aspartame 0.2%, almond powder 0.2%, and colorant.

Example 19: tartaric acid 3%, citric acid 12%, calcium carbonate 40%, baking soda 5%, taurine 23%, natural caffeine 2%, ginseng extract 10%, PEG 4%, vitamin E acetate 0.5%, stevioside 0.2%, mint flavor 0.2%, and colorant.

3. Description for Oral Health Effervescent Products

There are many oral healths, dental care, and breath refreshing products, like chewing gum, mouth wash and oral candy, etc. These products satisfied the needs of some consumers, but chewing a gum for a long time will lead to discomfort of jug maxillary muscle and lower jaw joint. Moreover, the gum base of a chewing gum is typically spit out which causes pollution to the environment. Mouth wash is not easy to carry and use and when used alcohol in mouth wash will stimulate oral mucosa causing irritation of your mouth. Moreover, it's not suitable to refresh your breath by using the products in a public event.

The present oral health effervescent product according to one embodiment provides oral health effervescent mint, tablets and edible chewing gum. They are convenient to carry and take than traditional effervescent tablets, and there is no need for water to dissolve for administration. When taking the product, it generates bubbles and water in mouth with a pleasant taste, refreshes breath, helps to restore a proper alkaline/acid balance of pH, prevents oral cavity and gum infection, and cleans teeth.

Therapeutic agents such as xylitol in the products are widely used in dentistry, unlike most other sugars, xylitol is non-fermentable and therefore cannot be converted to acids by oral bacteria, it has been shown to be beneficial for oral health and decrease dental cavities effectively. Xylitol can help prevent dental ulcer, gingivitis and periodontal disease. Coenzyme Q10 and ascorbic acid are antioxidants which can effectively prevent and cure gingival atrophy, dental bleeding, and oral infection. The present oral health effervescent products provide remarkable benefits to lower the risk of developing thrush, bacteria, fungal or yeast infection that can cause mouth soreness.

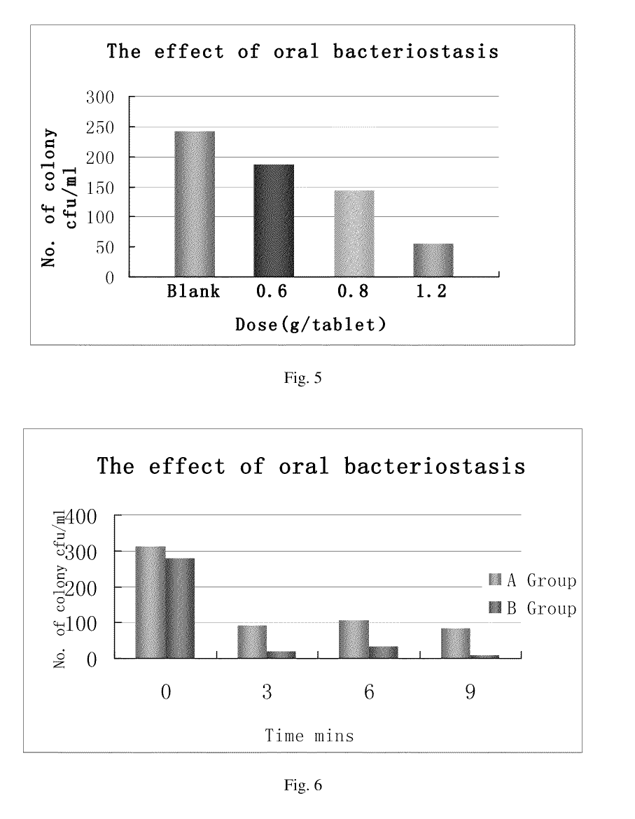

After taking one oral health effervescent mint in two minute with different dosage; 0.6 g, 0.8 g and 1.2 g separately, the comparison result of the effect of oral bacteriostasis is showed in FIG. 5: The effect of oral bacteriostasis by the comparison of different time; 3 minute, 6 minute and 9 minute separately is showed in FIG. 6. The results indicate the reduction of oral bacteria in two minute by taking oral health effervescent mint, and the more dosage to take the better results get. There is not much different effect by the time increasing and indicated that one oral health effervescent mint (0.8 g dosage) can reduce oral bacteria by more than ⅔ in two minutes.

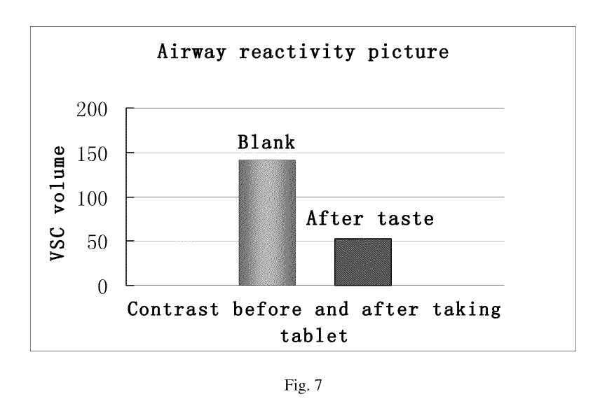

Detection of dispel halitosis by using a special breath testing equipment is shown in FIG. 7 which shows the effect of dispel halitosis by taking one oral health effervescent mint (0.8 g dosage) for 1 minute. The result indicated great reduction of halitosis and bad breath, oral health effervescent mint can refresh breath by almost ⅓ in two minutes.

The following examples are to illustrate the preparation method, but preparation methods are not limited to these.

### EXAMPLE

Example 1 provides a representative solid mixture formulation which is representative of compositions of the present invention; a sugar free, oral health effervescent mint includes: an edible basic formulation that comprises at least one component selected from the group consisting of alkali metal carbonates, alkali metal bicarbonates, alkaline earth metal carbonates, alkaline earth metal bicarbonates, pearl powder and mixtures thereof; and an edible acid formulation comprises at least one component selected from the group consisting of tartaric acid, citric acid, fumaric acid, maleic acid, malic acid, ascorbic acid, folic acid, acid phosphate salts, and mixtures thereof wherein the edible basic formulation and the edible acid formulation are in the range of from about 5 to about 50 percent by weight of the oral health effervescent mint, and xylitol in an amount of from about 10% to about 30%; coenzyme Q10 in an amount of from about 2% to about 10%; ascorbic acid in an amount of from about 5% to about 25%; PEG and or PVP in an amount of from about 5% to about 30%; flavoring mint in an amount of from about 0.2% to about 5%; and at least one excipient selected from the group consisting of sweeteners, coloring agents, processing aids.

Method of Preparation

Ingredients selected are mixed and are compressed into a tablet using a suitable effervescent system that includes basic components (sodium bicarbonate, sodium carbonate, calcium carbonate, but not limited to these) with a suitable acidic components (malic, citric, tartaric, but not limited to these). Typically one uses a 1:1 ratio of basic components to acidic components and total amount of about 10% to about 30%. In addition, an amount of about 5% to about 30% of therapeutic agents are mixed in the formulation; to assist in tablet disintegration about 0.5% to about 35% of processing material must be used such as PEG, PVP. Ingredients are mixed then compressed using a suitable tablet press under conditions of low humidity-less than 35% relative humidity. Additional excipient such as sweeteners, flavoring and coloring agents must be used to help compression.

The following examples are to illustrate the preparation method of oral health effervescent products including tablets, pills, granules, chewing gums, candy, but preparation methods are not limited to these.

Example 1: citric acid 5%, baking soda 15%, calcium carbonate 15%, xylitol 1%, PEG 35%, aspartame 0.3%, mint flavor 0.4%, sorbitol 5micro silicon 3%, and colorant.

Example 2: malic acid 40%, maleic acid 10%, sodium carbonate 5%, xylitol 40%, PEG 0.5%, stevioside 0.3%, hawthorn powder 0.9%, CMC 3%, dolomol 0.15%, and colorant.

Example 3: tartaric acid 10%, calcium carbonate 30%, coenzyme Q10 20%, glycerin monostearate 20%, PEG 9%, spicy powder 0.8%, mannitol 9%, stevioside 0.2%, talc powder 0.95%, and colorant.

Example 4: fumaric acid 10%, citric acid 20%, pearl powder 10%, xylitol 20%, vitamin C 5%, PEG 5%, panthenol 20%, sucralose 0.1%, mint flavor 0.35%, dextrin 9.5%, and colorant.

Example 5: tartaric acid 8%, citric acid 15.5%, baking soda 10%, calcium carbonate 16.5%, xylitol 16%, coenzyme Q 10 2%, PEG 12%, glycerin monostearate 5%, sorbitol 8%, sucralose 0.2%, flavoring strawberry essence 0.25%, PVP 1%, dolomo 10.5%, and colorant.

Example 6: citric acid 10%, folic acid 15%, pearl powder 35%, vitamin C 15%, glycerin monostearate 15%, orange powder 2.5%, saccharose 0.2%, lactose 5%, and colorant.

Example 7: citric acid 35%, calcium carbonate 40%, coenzyme Q 10 5%, PEG 15%, hawthorn powder 2.5%, neotame 0.1%, avice 2%, and colorant.

Example 8: adipic acid 15%, tartaric acid 25%, baking soda 45%, xylitol 2%, vitamin C 8%, panthenol 4%, aspartame 0.2%, almond powder 0.2%, talc powder 0.55%, and colorant.

Example 9: tartaric acid 15%, calcium carbonate 20%, xylitol 35%, PEG 22%, stevioside 0.2%, mint flavor 0.1%, sorbitol 7micro silicon 0.65%, and colorant.

Example 10: calcium carbonate 45%, baking soda 5%, pearl powder 10%, xylitol 10%, vitamin C 10%, PEG 10%, sucralose 0.3%, flavoring strawberry essence 0.5%, glycerol 6%, gelatin 3%, and colorant.

Example 11: malic acid 50%, sodium carbonate 5%, coenzyme Q 10 40%, PEG 0.5%, stevioside 0.3%, hawthorn powder 1%, vitamin E acetate 3%, and colorant.

Example 12: maleic acid 5%, baking soda 35%, calcium carbonate 15%, xylitol 1%, PEG 35%, aspartame 0.3%, mint flavor 0.6%, sorbitol 7%, edible oil 1%, and colorant.

Example 13: tartaric acid 10%, calcium carbonate 30%, coenzyme Q 10 20%, panthenol 15.5%, PEG 15%, glycerol 8%, sucralose 0.4%, flavoring lemon essence 0.6%, and colorant.

Example 14: fumaric acid 10%, malic acid 20%, pearl powder 5%, baking soda 25%, xylitol 25%, glycerin monostearate 5%, edible oil 5%, gelatin 3%, sucralose 0.1%, mint flavor 0.35%, and colorant.

Example 15: tartaric acid 5%, citric acid 15%, baking soda 10%, calcium carbonate 16%, vitamin C 30%, panthenol 1%, PEG 21.5%, Vitamin E acetate 1%, neotame 0.2%, flavoring strawberry essence 0.25%, and colorant.

Example 16: citric acid 10%, tartaric acid 16%, calcium carbonate 15%, xylitol 16%, glycerin monostearate 18%, orange powder 0.4%, saccharose 1%, glycerol 3.5%, gelatin 5%, and colorant.

Example 17: citric acid 35%, calcium carbonate 40%, coenzyme Q 10 5%, PEG 10%, gelatin 8%, edible oil 1.5%, aspartame 0.3%, coffee powder 0.1%, and colorant.

Example 18: adipic acid 17%, tartaric acid 25%, baking soda 20%, xylitol 5%, vitamin C 8%, panthenol 15%, mannitol 9.5%, aspartame 0.2%, almond powder 0.2%, and colorant.

Example 19: tartaric acid 15%, calcium carbonate 45%, xylitol 35%, PEG 4%, vitamin E acetate 0.5%, stevioside 0.2%, mint flavor 0.2%, and colorant.

4. Description for Nicotine Replacement Effervescent Products

Nicotine replacement therapy is the first choice solution to quit smoking. Nicotine replacement therapy uses a small amount of nicotine, which is less than tobacco, to replace nicotine in tobacco and reduce tobacco addiction. After a period of time, smokers need much less nicotine, and then give up smoking.

Nicotine replacement products include: nicotine transdermal patch, nicotine gum, nicotine nasal spray, nicotine inhalation. These products provide some ways that satisfy some smoker addition to nicotine in order to reduce part of their desire to smoke. But these products share a common disadvantage: it takes effect slowly. Nicotine transdermal patch will cause skin allergies, controlling the dosage is not easy, and sleep is affected if used at night. Chewing nicotine gum for a long time will lead to discomfort of the jugomaxillary muscle and the lower jaw joint, and the gum base of a chewing gum will cause pollution when it is discarding. Using nicotine chewing gum and candy in which nicotine absorbed by the gastrointestinal tract will be reduced by first-pass elimination and cause stimulus to gastrointestinal mucosa. It takes effect slowly and has low bioavailability. Nicotine nasal spray and inhalation is low in dosage and takes effect gradually. Patients use them frequently which could stimulate nasal, oral and threaten mucosa. These disadvantages will lead to the reduction of drug compliance, and eventually lead to the failure of therapy. It usually fails to make smokers resist tobacco use, and they smoke again.

To avoid the disadvantages of nicotine replacement products on the markets, the present invention including nicotine replacement effervescent tablets and nicotine replacement chewing candy, that are absorbed by bucal, sublingual and gingiva mucosa. Sublingual administration is second only to injection; it could maximize nicotine concentration in the blood and have a therapeutic effect. Nicotine replacement effervescent preparation is more convenient, more rapid, safer, and more effective than other nicotine replacement productions. It's the best fit for nicotine, and effectively relieves the pain of tobacco addiction. Nicotine replacement effervescent preparation can quickly aid in giving up smoking and refresh breath. The present invention provides a convenient, rapid and safe way of nicotine administration and greatly improves compliance. Administration of nicotine replacement effervescent does not require water, and does not interrupt social activity or draw other people's attention. The tablets do not only promote smoke abstinence, but also refreshes breath, which makes the present invention an ideal new medication for nicotine replacement therapy.

The following examples are to illustrate the preparation method, but preparation methods are not limited to these.

### EXAMPLE

Example 1 provides a representative solid mixture formulation which is representative of compositions of the present invention; a nicotine replacement (smoking cessation) effervescent tablet comprising: a edible basic formulation comprises at least one component selected from the group consisting of alkali metal carbonates, alkali metal bicarbonates, alkaline earth metal carbonates, alkaline earth metal bicarbonates, pearl powder and mixtures thereof; and an edible acid formulation comprises at least one component selected from the group consisting of tartaric acid, citric acid, fumaric acid, maleic acid, malic acid, ascorbic acid, folic acid, acid phosphate salts, and mixtures thereof, wherein the edible basic formulation and the edible acid formulation are in the range of from about 5 to about 50 percent by weight of the oral health effervescent mint; and nicotine in an amount of from about 0.01% to about 15%; liquorice root (radix gly-cyrrhizae) extract in an amount of from about 0.01% to about 15%; bomeol in an amount of from about 0.01% to about 15%; xylitol in an amount of from about 0.1% to about 35%; polyethylene glycol in an amount of from about 1% to about 35%; at least one artificial sweetener; and at least one flavoring and coloring agents.

Method of Preparation

Ingredients selected are mixed and are compressed into a tablet using a suitable effervescent system comprised of basic components (sodium bicarbonate, sodium carbonate, calcium carbonate, but not limited to these) with a suitable acidic components (malic, citric, tartaric, but not limited to these). Typically one uses a 1:1 ratio of basic components to acidic components and total amount of about 10% to about 30%. In addition, an amount of about 5% to about 30% of therapeutic agents are mixed in the formulation; to assist in tablet disintegration about 0.5% to about 35% of processing material must be used such as PEG, PVP. Ingredients are mixed then compressed using a suitable tablet press under conditions of low humidity-less than 35% relative humidity. Additional excipient such as sweeteners, flavoring and coloring agents must be used to help compression.

The following examples are to illustrate the preparation method of nicotine replacement products including tablets, pills, granules, chewing gums, candy, but preparation methods are not limited to these.

Example 1: citric acid 5%, baking soda 35%, calcium carbonate 15%, nicotine 1%, PEG 35%, aspartame 0.3%, mint flavor 0.6%, sorbitol 4micro silicon 1%, and colorant.

Example 2: malic acid 50%, sodium carbonate5%, liquorice root extract powder 39.8%, nicotine 0.2%, PEG 0.5%, stevioside0.3%, hawthorn powder0.9%, PVP3%, dolomol 0.15%, and colorant.

Example 3: tartaric acid 10%, calcium carbonate 30%, nicotine 10%, borneol 10%, panthenol 16%, PEG15%, spicy powder 0.8%, mannitol 7%, stevioside 0.2%, talc powder 0.95%, and colorant.

Example 4: fumaric acid 10%, citric acid 20%, baking soda 5%, pearl powder 5%, nicotine 5%, borneol 5%, liquorice root extract powder 15%, PEG 5%, glycerin monostearate 20%, sucralose 0.1%, mint flavor 0.35%, dextrin 9.5%, and colorant.

Example 5: tartaric acid 7%, citric acid 15%, baking soda 10.5%, 17.5%, nicotine 0.1%, borneol 9.9%, ginseng extract 20%, PEG 12%, panthenol 7%, sucralose 0.2%, flavoring strawberry essence 0.25%, dolomol 0.5%, and colorant.

Example 6: citric acid 10%, folic acid 15%, pearl powder 35%, nicotine 0.5%, borneol 14.5%, glycerin monostearate 15%, orange powder 2.9%, saccharose 2%, lactose 5%, and colorant.

Example 7: citric acid 35%, calcium carbonate 40%, nicotine 2%, liquorice root extract powder 3%, PEG 13%, hawthorn powder 4.5%, neotame 0.2%, avicel 2%, and colorant.

Example 8: maleic acid 15%, tartaric acid 25%, baking soda 45%, nicotine 3%, borneol 7%, panthenol 4%, aspartame 0.2%, almond powder 0.2%, talc powder 0.55%, and colorant.

Example 9: tartaric acid 5%, adipic acid 10%, calcium carbonate 12%, baking soda 8%, nicotine 4%, borneol 6%, liquorice root extract powder 25%, PEG 22%, stevioside 0.2%, flavoring lemon essence 0.1%, sorbitol 7micro silicon 0.65%, and colorant.

Example 10: calcium hydrogen phosphate 45%, baking soda 5%, pearl powder 10%, nicotine 8%, ginseng extract 7%, PEG15%, sucralose0.3%, flavoring strawberry essence 0.5%, glycerol 16%, gelatin 3%, and colorant.

Example 11: malic acid 50%, sodium carbonate 5%, nicotine 8%, liquorice root extract powder 32%, PEG 0.5%, stevioside 0.3%, hawthorn powder 1%, vitamin E acetate 3%, and colorant.

Example 12: maleic acid 5%, baking soda 30%, calcium carbonate 20%, nicotine 1%, PEG 35%, aspartame 0.3%, mint flavor 0.6%, sorbitol 7%, edible oil 1%, and colorant.

Example 13: tartaric acid 15%, calcium carbonate 25%, nicotine 6%, borneol 4%, liquorice root extract powder 10%, panthenol 15.5%, PEG 15%, glycerol 8%, sucralose 0.4%, flavoring lemon essence 0.6%, and colorant.

Example 14: fumaric acid 10%, malic acid 20%, pearl powder 5%, baking soda 25%, nicotine 9%, borneol 16%, glycerin monostearate 5%, edible oil 5%, vitamin E acetate 1.5%, gelatin 3%, sucralose 0.1%, mint flavor 0.35%, and colorant.

Example 15: tartaric acid 7%, citric acid 15%, baking soda 10%, calcium carbonate 13%, pearl powder 5%, nicotine 1%, borneol 1%, liquorice root extract powder 28%, panthenol 1%, PEG17.5%, vitamin E acetate 1%, neotame 0.2%, flavoring strawberry essence 0.25%, and colorant.

Example 16: citric acid 10%, tartaric acid 15%, calcium carbonate 17%, pearl powder 18%, nicotine 0.05%, borneol 4.5%, liquorice root extract powder 5%, glycerin monostearate 20%, orange powder 0.4%, saccharose 1%, glycerol 3.5%, gelatin 5%, and colorant.

Example 17: citric acid 35%, calcium carbonate 40%, nicotine 5%, PEG 10%, gelatin 8%, edible oil 1.5%, aspartame 0.3%, coffee powder 0.1%, and colorant.

Example 18: adipic acid 17%, tartaric acid 23%, baking soda 20%, nicotine 0.2%, borneol 0.5%, liquorice root extract powder 14.3%, panthenol 15%, mannitol 9.5%, aspartame 0.2%, almond powder 0.2%, and colorant.

Example 19: tartaric acid 3%, citric acid 12%, calcium carbonate 40%, baking soda 5%, nicotine 2%, borneol 3%, liquorice root extract powder 30%, PEG 4%, vitamin E acetate 0.5%, stevioside 0.2%, mint flavor 0.2%, and colorant.

While the invention has been described and illustrated in connection with preferred

While the foregoing invention has been described in some detail for purposes of clarity and understanding, it will be appreciated by one skilled in the art, from a reading of the disclosure that various changes in form and detail can be made without departing from the true scope of the invention.

What is claimed is:

1. A solid effervescent mixture for oral absorption in solid dosage form, including at least one of tablets, pills, granules, chewing gums, and candy.

2. The solid effervescent mixture for oral absorption according to claim 1, comprising at least one of therapeutic agent, wherein no water is needed to dissolve the effervescent mixture prior to administration and to help swallowing, and when administered orally the solid effervescent mixture generates bubbles and water.

3. The solid effervescent mixture for oral absorption according to claim 1, comprising a basic component selected from a group consisting of alkali metal carbonates, alkali metal bicarbonates, alkaline earth metal carbonates, alkaline earth metal bicarbonates, pearl powder and their mixture, wherein the basic component is in a range of about 5% to about 50% by weight of the mixture.

4. The solid effervescent mixture for oral absorption according to claim 1, comprising an acidic component selected from a group consisting of tartaric acid, citric acid, adipic acid, fumaric acid, maleic acid, malic acid, ascorbic acid, folic acid, acid phosphate salts and their mixture, wherein the acidic component is in the range of about 5% to about 50% by weight.

5. The solid effervescent mixture for oral absorption according to claim 1, comprising at least one therapeutic agent selected from a group consisting of an analgesic, an herbal and dietary supplement, an oral health or dental care agent, an energy enhancing agent, an anti-smoke agent and a sober-up formula alcoholism.

6. The solid effervescent mixture for oral absorption according to claim 1, comprising at least one processing material selected from the group consisting of PEG (polyethylene glycol), PVP (polyvinylpyrrolidone), panthenol, vitamin E acetate, glycerin monostearate and their mixture, wherein the at least one processing material is in the range of about 1% to about 35% by weight.

7. The solid effervescent mixture for oral absorption according to claim 1, comprising at least one excipient selected from a group consisting of a sweetener, edible oil, a flavoring or coloring agent, a coating material, processing aid, and a breath freshener, wherein the at least one excipient is in the range of about 0.1% to about 15% by weight.

8. The solid effervescent mixture for oral absorption according to claim 5, wherein the therapeutic agent is a sober up formula comprising at least one component selected from a group consisting of glucurolactone, natural caffeine, extracts of hawthorn fruit, green tea, hovenia dulcis, nutmeg, poria cocos, fructus tsaoko, radix puerariae, semen ziziphi spinosae, ramulus cinnamoni, alpinia officinarum hance, and chrysanthemum, wherein the least one component in the therapeutic agent is in the range of about 1% to about 40% by weight of the mixture.

9. The solid effervescent mixture for oral absorption according to claim 5, wherein the therapeutic agents is an energy enhancing component comprising at least one component selected from a group consisting of ginseng extract, octacosanol, glucurolactone, taurine, rhodiola, natural caffeine and d-ribose, wherein the at least one component in the therapeutic agent is in the range of about 1% to about 30% by weight of the mixture.

10. The solid effervescent mixture for oral absorption according to claim 5, wherein the therapeutic agent in is an oral health or dental care component comprising at least one component selected from the group consisting of xylitol, ascorbic acid and Coenzyme Q10, wherein the at least one component in the therapeutic agent is in the range of about 1% to about 30% by weight; thereof in the therapeutic agent comprising more preferably in an amount of about 5% to about 25% by weight of the solid mixture

11. The solid effervescent mixture for oral absorption according to claim 5, wherein the therapeutic agent is a nicotine replacement and anti-smoking component comprising at least one component is selected from a group consisting of nicotine, liquorice root (radix gly-cyrrhizae) extract and borneol, wherein the at least one component in the therapeutic agent is in the range of from about 0.5% to about 30% by weight of the mixture.

12. A method for preparing the solid effervescent mixture for oral absorption having at least one therapeutic component, a basic component, and an acidic component, the method comprising:
a. granulating and passing each of the components through an 80 mesh screen separately,
b. mixing each of the components with a processing material separately,
c. melting each of the component and processing material mixtures separately in an heating oven separately,
d. lowering the temperature of each of the component and processing material mixtures separately after melt agglomeration,
e. pelletizing and sorting through a screen each of the component and processing material mixtures separately,
f. combining each of the pelletized components according to a solid formulation;
g. adding an appropriate amount of at least one of a sweetener, a flavoring agent and coloring agent, according to the different product requirement, and
h. processing the component mixture into one of tablets, pills and granules.

13. A method for preparing the solid effervescent mixture for oral absorption having at least one therapeutic component, a basic component, and an acidic component, the method comprising:
a. granulating and passing each of the components through an 80 mesh screen separately,
b. mixing each of the components with a processing material according to a solid formulation in a kneading machine,
c. heating the mixture;
d. adding an appropriate amount of at least one of a sweetener, a flavoring agent, a coloring agent, and a processing agent,
e. processing the mixture into a softwood sheet,
f. cutting and shaping the mixture into individual items, and
g. coating the items with coating materials to make chewing gum and candy.
